(19) United States
(12) Patent Application Publication
(10) Pub. No.: US 2001/0014343 A1
(43) Pub. Date: Aug. 16, 2001

(54) Skin care composition

(76) Inventor: Marie Harbeck, Austin, TX (US)

Correspondence Address: MARIE HARBECK, 3202 CLUMPARASS COVE, AUSTIN, TX, 78735, US

(21) Appl. No.: 09/753,902

(22) Filed: Jan. 3, 2001

Related U.S. Application Data
(63) Continuation-in-part of application No. 09/248,573, filed on Feb. 11, 1999, now Pat. No. 6,193,987.

(51) Int. Cl. A61K 7/06

(52) U.S. Cl. 424/401

(57) ABSTRACT

Improved and useful formulation for a topical transdermal delivery system in the form of a lotion for the swift alleviation and treatment of skin conditions such as dermatitis, eczema, lupus, and rosacea, including cracking, peeling, flaking, dry and distressed skin infirmities, that has as its constituents a mixture of safflower oil, flaxseed oil, tincture of benzoin, vitamin A and tocopheryl linoleate.

This invention is a “Continuation-In-Part” application for Ser. No. 09/248,573 filed Feb. 11, 1999 for Marie Harbeck.

### BACKGROUND-FIELD OF INVENTION

This invention relates to improved and useful formulation for a topical transdermal skin care composition for alleviating and treating skin infirmities, including dermatitis, eczema, lupus, rosacea, and dry skin, that has as its main consituents a mixture of organic safflower oil, flaxseed oil, and tincture of benzoin.

To the best of the applicant's knowledge the following is the most relevant prior art:

U.S. Pat. No. 4,740,367 Force, et al. 1988

U.S. Pat. No. 5,244,679 Freston. 1993

U.S. Pat. No. 5,489,429 Griat, et al. 1996

U.S. Pat. No. 5,683,704 Ohba, et al. 1997

U.S. Pat. No. 4,883,659 Goodman, et al. 1989

U.S. Pat. No. 4,608,392 Jacquet et al. 1986

U.S. Pat. No. 5,431,911 Reynolds. 1995

The cosmetic and pharmaceutical industry is continually expanding its efforts in order to provide topical preparations which will moisten and soften the skin, eliminate dryness, cracking, chapping, chaffing, redness and restore the skin to its natural healthy condition.

Thus, synthetic surfactants, silicones, ammonia, alcohol's, solvents, acids, artificial colors and fragrances, and the like are being incorporated into the compositions of the cited prior art to extend shelf life of the cited compositions that are supplied to stores and supermarkets, some of these components aggravate skin infirmities thus the combinations of skin aggravating synthetic compositions employed continue to increase yearly.

It will be appreciated that the expansion and growth of the cited prior art has devolved into highly complex synthetic adjuvants for use in the formulations.

The problem with the use of the compositions of the cited prior art which make up the bulk of transdermal delivery systems are their chemical structures which are known to cause hand, foot, scalp and skin irritations.

Alcohol, acids, solvents, synthetic perfumes and colorants, and ammonia adjuvants incorporated into the compositions of the prior art cause dryness of the skin, rashes, allergies, dermatitis, peeling, bleeding, itching, painful cracking and flaking of the skin.

Unfortunately, the cited prior art compositions are relatively ineffective for distressed skin conditions, not allowing the treatment and alleviation of skin irritations related to allergic skin reactions to synthetic adjuvants, so there is a need in the art for organic compounds for improved skin care compositions.

The said prior art compositions do not employ the massive quantities of cold-pressed organic vegetable oils.

The said prior art compositions do not employ organic beeswax compositions that this invention incorporates due to prohibitive manufacturing costs.

In terms of effectiveness, the said cited prior art compositions do not provide satisfactory skin penetration and supplying properties to leave skin satiny smooth for up to twelve hours.

The said cited prior art compositions do not provide the high volume of cold pressed organic safflower oil, or flaxseed oil, in their formulas to restore essential oil lipids that are essential to nourish and maintain healthy skin.

The said cited prior art compositions do not provide the high volume of skin healing vitamin A and tocopheral linoleate compositions to their formulations due to the prohibitively expensive manufacturing costs.

The said cited prior art compositions do not boil tincture of benzoin to eliminate its alcohol content.

The said cited prior art compositions do not provide an urgent need that exists for compositions like the present invention to the dermatological, pharmaceutical, alternative therapy and cosmetology fields.

The said cited prior art compositions do not provide organic compositions to alleviate and treat the symptoms of lupus and rosacea skin infirmities.

The said cited prior art compositions do not provide organic compositions to cool and calm skin after micro-dermabrasion, salicylic, glycolic and alpha-hydroxy acid peels, after tattoo removal and laser skin treatments.

The said cited prior art compositions do not provide organic moisturizing compositions that provide a water resistant, non-greasy, antiseptic film on the skin.

The said cited prior art compositions do not provide organic compositions that are water resistant to body fluids, sebaceous gland activity and perspiration.

The said cited prior art compositions do not provide organic compositions to provide a high volume of organic essential oils for anti-inflammatory qualities and to perfume the said compositions.

The said cited prior art compositions do not provide a high volume of organic safflower and flaxseed oils in the said compositions.

The said cited prior art compositions do not provide high volume organic compositions to replace lost oil lipids to the dermis.

The said cited prior art compositions do not provide gentle, organic compositions for infant and adolescent use.

### OBJECTIVES AND ADVANTAGES OF THE PRESENT INVENTION ARE

a) to provide an alcohol free topical skin preparation,

b) to provide an essential plant oils as an organic antiseptic adjuvant to treat, bacterial, fungal and environmental aggressors of the skin,

c) to provide a topical skin composition that has potent effectiveness in the treatment of skin infirmities, such as dermatitis, eczema, lupus, rosacea, chapping, rough skin, dryness, itching, cracking, flaking, and skin allergies related to synthetic substances and the like;

d) to provide a high volume of organic cold pressed vegetable oil compositions for immediate transdermal delivery,

e) to provide a satisfactory skin penetration of the said composition and supplying properties that leave the skin satiny smooth for up to twelve hours,

f) to provide a high volume of organic essential oils for anti-inflammatory qualities and to perfume the said composition,

g) to provide a high volume of organic safflower and flaxseed oils in the said composition to replace oil lipids to the dermis,

h) to provide an urgent need that exists for composition like the present invention to the dermatological, pharmaceutical, alternative therapy and cosmetology fields,

i) to alleviate and treat the symptoms of dermatitis, rough skin, dryness, cracking, eczema, chapping, itching and allergic skin reactions due to ammonia, solvents, acids, steroid, alcohol substances, and synthetic perfumes and colorants;

j) to provide an organic beeswax substance that bonds lubricating, hydrating, moisturizing and antiseptic compounds to the skin for up to twelve hours;

k) to provide an organic calming, cooling, moisturizing composition to the skin after micro-dermabrasion, salicylic, glycolic, alpha-hydroxy acid peels, after tattoo removal, and laser skin treatments;

l) to provide gentle, organic composition for infant and adolescent use,

m) to provide an organic water resistant, non-greasy, antiseptic and moisturizing film on the skin,

n) to provide an organic composition that is water resistant to body fluids, sebaceous gland activity and perspiration;

o) to provide an organic composition without chemical structures that are known to cause skin irritations, and

p) to provide an organic composition without solvents, artificial perfumes, artificial colorants and ammonia additives;

Thus, an urgent need exists for skin composition like the present invention, that employs a high volume of potent cold-pressed organic flaxseed and safflower oils plus organic vitamins to feed the skin with nutritive polyunsaturated oil lipids without the addition of skin irritating synthetic adjuvants to achieve its purposes. Further advantages of this invention are the employment of an organic wax substance and an organic antiseptic substance that bonds to the skin to protect against and to fight environmental skin aggressors, leaving the skin moisturized and in a healthy condition for up to twelve hours. Still further objects and advantages will become apparent from a consideration of the ensuing descriptions.

### BRIEF SUMMARY OF THE INVENTION

A topical skin composition supplying essential oil lipids for the swift alleviation and treatment of dry skin conditions, including dermatitis, eczema, lupus, rosacea, rough skin, cracking, itching, peeling, redness, allergic skin reactions to alcohol and solvent sensitivity, which has as its main consituents, tincture of benzoin, safflower oil, flaxseed oil, vitamin A, tocopheral linoleate, organic beeswax, glycerin, borax, stearic acid, perfuiming ingredients such as essential oil of rosemary, oil of orange and oil of pepperming, and water.

### DESCRIPTION OF THE INVENTION

It is an objective of the improved invention to provide a potent, swift acting, non-sticky, organic, transdermal delivery formulation for treating and alleviating dry, distressed skin conditions, including dermatitis and eczema, lupus, rosacea, rough skin, cracking, itching, peeling, redness, allergic skin reactions to alcohol and solvent sensitivity.

It is an objective of the improved invention to provide an improved formulation which soothes, moisturizers and restores essential nutrients to the skin to improve its texture and protect it against dryness.

It is still a further objective of the invention to provide an organic formulation for the treatment, alleviation and maintenance of cracked, callused, rough, skin of the feet.

These and other objects are accomplished in accordance with a particular embodiment of the present improved invention which provides a skin care composition in the form of a lotion, comprising a mixture consisting of tincture of benzoin, vitamin A, tocopheral linoleate, organic beeswax, cold-pressed flaxseed, safflower oils, glycerin, borax, stearic acid, and essential plant oils and mixtures thereof The essential components of the present improved composition readily intermix to form an improved mixture suitable for topical applications to the skin. The present components are combined in such relative proportions so as to bring about a marked beautifying effect to the skin by supplying to the skin nutrients that are essential for healthy skin.

The present improved invention includes the surprising discovery that cold-pressed organic safflower and flaxseed oils, tincture of benzoin, high volumes of vitamins A and E, can be used as active principals in the improved skin care composition to treat conditions attributed to lupus, rosacea, eczema and distressed skin conditions. In accordance with the present improved invention there is provided a formulation where cold-pressed, organic safflower oil, flaxseed oil, and tincture of benzoin can be employed with organic beeswax to aid in the water-resistant, non-greasy treatment and alleviation of chronic skin conditions. The embodiment of the improved invention follow to illustrate the formulation.

The main ingredients of this lotion are:

0.5 to 2000 milliliters water,

2.5 to 800 grams stearic acid,

0.2 to 800 grams cetearyl alcohol & polysorbate 60 called LipowaxP, purchased from Lipo Chemicals Inc, 207 19th Avenue, Paterson, N.J. 07504,

0.2 to 800 milliliters sweet almond oil,

0.5 to 900 milliliters cold-pressed safflower oil,

0.2 to 500 milliliters apricot kernel oil,

0.5 to 500 grams cocoa butter,

0.5 to 800 grams organic white beeswax,

0.2 to 800 milliliters jojoba oil,

0.1 to 900 milliliters glycerin,

0.01 to 800 milliliters cold-pressed flaxseed oil,

0.001 to 600 milliliters tocopheral linoleate,

0.1 to 80 milliliters composition containing 30000 micrograms vitamin A/ml,

0.2 to 600 milliliters essential oil of rosemary,

0.1 to 600 milliliters essential oil of peppermint,

0.1 to 600 milliliters essential oil of orange,

0.1 to 200 grams borax granules,

0.001 to 80 milliliters tincture of benzoin, and

0.2 to 100 milliliters dmdm hydantoin,

Therefore my lotion process is as follows:

place safflower oil, sweet almond oil, apricot kernel oil, tocopheral linoleate, vitamin A, jojoba oil, flaxseed oil, cetearyl alcohol and polysorbate 60, beeswax, stearic acid, cocoa butter and glycerin in a partially submerged container over hot water to form a mixture, stirring said mixture over hot water until dissolved;

simultaneously placing water, borax and tincture of benzoin, in another partially submerged container over hot water to form a mixture; stirring said mixture over hot water until dissolved,

removing said mixtures from heat and blending both said mixtures until said second mixtures cool to 35 degrees C;

adding essential oils of rosemary, peppermint

and orange oils, and dmdm hydantoin to mixture;

and blending mixture until it thickens,

whereby the ingredients are formed into a cream base.

It is believed that the antiseptic, protective, softening, long term lubricating properties and the high volume of organic cold-pressed flaxseed oil lipids, and organic cold-pressed safflower oil lipids, and the high volume of vitamins A and tocopheral linoleate, and tincture of benzoin, emulsified in organic beeswax are responsible for the alleviation, and treatment of dermatitis, emulsified in organic beeswax are responsible for the alleviation, and treatment of dermatitis, eczema, rosacea, lupus, fungal infections, and damaged skin, but we don't wish to be bound by this.

As noted, the improved composition hereof comprises a water-resistant, topical application which is applied to the dermal areas of the body to alleviate and treat rosacea, lupus, dry, chapped, flaky, rough, reddened, cracked, and chronic dry skin conditions, by feeding skin the essential polyunsaturated oil lipids it needs to maintain its healthy condition.

This improved formulation is highly effective for cosmetic and pharmaceutical use in the treatment of lupus, dermatitis, eczema and dry skin due to the resistance of the formulation to attack by body liquids, sebaceous oils and perspiration.

This improved formulation provides a water-resistant, light-weight lotion that is preferred to be used during the daytime as a moisturizer and skin protectant.

This improved formulation employs organic polyunsaturated oil lipids in the place of mineral oil, which by its solvent action strips skin surface lipids.

This improved formulation moistens the skin in an invisible manner, thereby eliminating unsightly flaky dead skin cells which result from chemical peels.

For a more complete understanding of the present improved composition reference is made to the following examples. The following examples are illustrative of the present improved composition and are not intended in any as a limitation upon the scope thereof

### EXAMPLE 1

The composition of the lotion was applied twice daily to the face and body of a woman suffering from lupus, which results in chronic, painful, dry skin. Within two days of use, the skin became soft and pliable, and the softening effects lasted all day.

A female user, suffering from a red, raised, itchy, rash from medication allergies, found the lotion gave immediate, cooling relief from the itch and within three days, the rash and redness were gone.

### EXAMPLE 2

To evidence the efficacy of the improved composition, a series of individuals suffering from dry skin, were treated with the improved lotion formulation. Compared to commercially available skin moisturizers, the results based upon the efficacy of the present invention are set forth hereinafter:

a) male user found the present invention alleviated dry, flaky skin cause by air-conditioning;

b) female user found the present invention moisturized and soothed her itching skin due to sun exposure;

c) teenage female user found the present invention did not strip the natural oils from her skin compared to commercially available skin lotions;

d) the present invention was used by a female dermatitis sufferer, who found that the skin on her hands did not crack compared to previously used commercially available lotions.

### EXAMPLE 3

A female user found that twice daily applications of the lotion, healed her cracked heels and feet, after which, a daily application kept her feet and heels, smooth, nourished and crack free.

### EXAMPLE 4

Male and female users reported that the lotion cooled, soothed and alleviated their hot, painful, sun burnt skin, and the cooling effects lasted for up to an hour after each application.

### EXAMPLE 5

Male and female found that the present improved invention, did not aggravate their dermatological infirmities, and much to their delight, left their skin soft, comfortable, and pleasantly perfumed with invigorating aromatherapeutic essential oils.

Thus, from the preceding it will be readily perceived by the reader that an urgent need exists for this improved composition for the alleviation of chronic skin conditions denoted heretofore, but which also, can be used to prevent such conditions from occurring;

The improved composition provides:

an alcohol, silicone and solvent free formulation;

organic antiseptic adjuvants to fight bacterial, fungal and environmental aggressors;

a topical composition that has immediate effectiveness in the treatment of chronic skin infirmities;

superior skin penetration of the said composition and nutrient supplying properties;

organic anti-inflammatory qualities;

high volumes of organic polyunsaturated oils to replace lost dermal lipids;

fills an urgent need in the pharmaceutical, alternative health, and cosmetic markets;

alleviates and treats allergic skin reactions to steroid and cortizone treatments;

organic beeswax to bond lubricating, hydrating, antiseptic and moisturizing compounds to the skin;

organic calming, cooling, moisturizing compounds to skin after acid peels, after tattoo removal treatment and laser treatments;

a composition that does not strip the skin of its natural oils;

a non-greasy moisturizing film on the skin that is resistant to attack by body fluids, sebaceous oils and perspiration;

a light-weight emulsion that is preferred for day time application;

organic oil lipids in the place of mineral oil, which by its solvent action strips skin surface lipids.

While the above description contains many specificity's, these should not be construed as limitations on the scope of the improved invention, but merely providing illustrations of some of the presently preferred embodiments of this improved invention.

Many other variations are possible. For example, it is additionally possible to add to the present invention many other adjuvants such as ultraviolet filters, color pigments, pearling pigments, additional essential plant oils and perfuming ingredients, vitamins, antibiotics, and additional plant and vegetable oils and be within the purview of this invention.

Accordingly, the scope of the invention should be determined not by embodiments illustrated, but by the appended claims and their legal equivalents, rather than by the examples given.

What is claimed is:

1. A lotion comprising:
from 0.2 to 800 milliliters of sweet almond oil,
from 0.2 to 800 milliliters of safflower oil,
from 0.2 to 800 milliliters of apricot kernel oil,
from 0.2 to 800 milliliters of flaxseed oil,
from 0.2 milliliters to 200 milliliters of tincture of benzoin,
from 0.2 milliliters to 600 milliliters of tocopheral lineolate,
from 0.5 to 2000 milliliters of water,
from 0.2 to 800 milliliters of jojoba oil,
from 0.2 to 800 grams cetearyl alcohol & polysorbate 60,
from 0.5 to 800 grams beeswax,
from 0.5 to 800 grams stearic acid,
from 0.5 to 500 grams cocoa butter,
from 0.1 to 80 milliliters composition containing 3000 micrograms vitamin A/ml,
from 0.2 to 900 milliliters glycerin,
from 0.1 to 200 grams powdered borax,
from 0.2 to 100 milliliters a preservative,
from 0.2 to 600 milliliters essential oil of peppermint,
from 0.2 to 600 milliliters essential oil of orange,
from 0.2 to 600 milliliters a perfuming oil.

2. A lotion comprising:
135 milliliters water,
15 grams stearic acid,
15 grams cetearyl alcohol,
10 milliliters sweet almond oil,
10 milliliters safflower oil,
10 milliliters apricot kernel oil,
7.5 grams cocoa butter,
5 grams beeswax,
5 milliliters jojoba oil,
5 milliliters glycerin,
10 milliliters cold-pressed flaxseed oil,
10 milliliters tocopheral linoleate,
10 milliliters composition containing 3000 micrograms vitamin A/ml,
1.25 milliliters a perfuming oil,
2.5 milliliters essential oil of peppermint,
2.5 milliliters essential oil of orange,
1.25 grams borax granules,
0.6 milliliters tincture of benzoin,
0.3 millilitres of a preservative such as dmdm hydantoin,

3. A method of making a lotion comprising:
adding in a container being heated while said container is partially submersed in a 100 degree C bath, safflower oil, sweet almond oil, apricot kernel oil, tocopheral linoleate, vitamin A, jojoba oil, flaxseed oil, cetearyl alcohol and polysorbate 60, beeswax, stearic acid, cocoa butter and glycerin to form a mixture,
stirring said mixture until dissolved;
adding simultaneously in second container while said second container is being heated while partially submersed in a 100 degree C bath, water, borax and tincture of benzoin,
stirring said mixture until mixture reaches 80 degrees C, to boil off alcohol content in tinture of benzoin,
stirring said mixture until dissolved;
removing said mixtures from heat and blending both mixtures until said mixtures cool to 40 degrees C;
adding essential oils of rosemary, peppermint and orange oils, and dmdm hydantoin, and
blending mixture until it thickens, whereby the ingredients are formed into a cream base.
